(19) United States
(12) Patent Application Publication
(10) Pub. No.: US 2004/0053968 A1
(43) Pub. Date: Mar. 18, 2004

(54) Methods and compositions for treating peridontal disease

(76) Inventors: George D. Hartman, Lansdale, PA (US); Sevgi B.. Rodan, Bryn Mawr, PA (US); Gideon A. Rodan, Bryn Mawr, PA (US)

Correspondence Address: MERCK AND CO INC, P O BOX 2000, RAHWAY, NJ, 070650907

(21) Appl. No.: 10/250,546

(22) PCT Filed: Dec. 28, 2001
(86) PCT No.: PCT/US01/49638

(51) Int. Cl. A61K 31/444; A61K 31/365; A61K 31/415; A61K 31/42

(52) U.S. Cl. 514/333; 514/406; 514/473; 514/378

(57) ABSTRACT

The present invention provides for a method for the treatment of alveolar bone loss due to periodontal disease in a subject in need of such treatment comprising administration of a therapeutically effective amount of an αvβ3 integrin receptor antagonist in combination with a therapeutically effective amount of a COX-2 inhibitor. Further, the present invention provides for pharmaceutical compositions useful in the methods of the present invention, as well as a method of manufacture of a medicament useful for treating the alveolar bone loss due to periodontal disease.

### FIELD OF THE INVENTION

The present invention provides for novel methods for the treatment of alveolar bone loss due to periodontal disease comprising a drug combination. More particularly, the drug combination of the present invention comprises an αvβ3 integrin receptor antagonist in combination with a selective cyclooxygenase-2 inhibitor. The present invention also provides for pharmaceutical compositions comprising such drug combinations useful in the methods to treat periodontal disease. Moreover, the present invention provides for a method of manufacture of a medicament useful in the treatment of periodontal disease.

### BACKGROUND OF THE INVENTION

Periodontal disease is a common chronic disease primarily of adults. It is characterized by inflammation and degeneration of the tissues that surround and support mammalian teeth. These include the gingiva (gums), periodontal ligament, and alveolar bone. Periodontitis, the inflammation of the gingiva and eventual loss of alveolar bone, is the latest stage of this progressive disorder and is the major cause of tooth loss in older adults. Localized juvenile periodontitis and early-onset periodontitis have similar presentation, outcome, and etiology, but present at younger ages. Identified causal factors for periodontitis include the presence of bacterial plaque that harbors actinobacillus actinomycetemcomitans and porphyromonas gingivalis. In 80% of individuals, the presence of these microorganisms leads only to gingivitis. However, risk factors that include smoking, diabetes, various immune deficiency disorders (autoimmune diseases, HIV) and dental mechanical abnormalities, such as malocclusion, malaligned teeth, and faulty dental restorations, have been identified that lead to most cases of periodontitis. The rate of bone loss (resorption) depends upon the severity of the above conditions or causative factors. Symptoms of periodontitis include the development of pockets between the gingivae and the teeth; loss of attachment of the gums and bone to the teeth; alveolar bone loss; tooth mobility; and tooth loss. Improved oral hygiene is the basis for current corrective measures of this disease. Supragingival scaling, then scaling and root planing to remove subgingival plaque and toxin-laden cementum, are the first line dental treatment measures for controlling periodontitis. More advanced forms of periodontal surgery are occasionally used to reduce pocket depths to facilitate home care. However, periodontal surgery itself may induce or, at a minimum, be associated with alveolar bone loss.

Antibiotics, astringent agents, and mouthwashes are not effective for long term treatment of periodontitis. Home care that includes brushing, rinsing, and flossing is effective at controlling periodontitis, when pocket depth is minimal. Other preventive treatments include hydrogen peroxide mouth rinses (3 percent H₂O₂ in warm water). Carbamide peroxide (urea hydrogen peroxide) is also used in the local treatment of minor infections and inflammation caused by periodontitis. Penicillin or erythromycin may be used if severe oral signs or symptoms are present. However, the antibiotic agents used to treat periodontal disease in the clinic at the present time are not very effective.

Periodontal disease progresses through bursts of bone destruction. The bone loss associated with periodontal disease, like that in other parts of the skeleton, results from an imbalance between bone resorption, i.e. breakdown, and bone formation. However, the rate of bone turnover (combined formation and resorption) differs from site to site; for example, it is higher in the trabecular bone of the vertebrae and the alveolar bone in the jaws than in the cortices of the long bones. The potential for bone loss is directly related to turnover and can reach over 5% per year in vertebrae immediately following menopause, a condition which leads to increased fracture risk. Periodontitis arises most often from the activation of local immune inflammatory mechanisms and results in the release of inflammatory mediators. The released mediators include various prostaglandin products of the cyclooxygenase pathway of arachidonic acid metabolism, such as prostaglandin E₂ (PGE₂) and prostaglandin I₂. In particular, PGE₂ has been implicated as a key inflammatory mediator in periodontal disease and has been shown in culture to cause decreased collagen synthesis in fibroblasts and to stimulate osteoclastic bone resorption [See R. Dziak, “Biochemical and Molecular Mediators of Bone Metabolism”, Journal of Periodontology, Vol. 64, pp. 407-415 (1993), which is incorporated by reference herein in its entirety]. Individuals suffering from periodontal disease may benefit from treatment with agents that inhibit alveolar bone resorption.

Non-steroidal, anti-inflammatory drugs (NSAIDs) exert most of their anti-inflammatory, analgesic and antipyretic effect through inhibition of prostaglandin G/H synthase, also known as cyclooxygenase. As prostaglandins have both physiological and pathological roles, evidence is mounting that the constitutive enzyme, cyclooxygenase-1 (COX-1), is responsible, in large part, for endogenous basal release of prostaglandins and hence is important in their physiological functions such as the maintenance of gastrointestinal integrity and renal blood flow. It is now well documented that the inducible cyclooxygenase (COX-2) is associated with inflammatory conditions, whereas COX-1 is responsible for cytoprotective effects of prostaglandins. In contrast, the inducible form, COX-2, appears to be mainly responsible for the role of prostaglandins in pathophysiology where rapid induction of the enzyme occurs in response to such agents as inflammatory agents, hormones, growth factors, and cytokines.

Inhibitors of COX-2 are a subclass of the class of drugs known as non-steroidal anti-inflammatory drugs (NSAIDs). The NSAIDs are active in reducing the prostaglandin-induced pain and swelling associated with the inflammation process but are also active in affecting other prostaglandin-regulated processes not associated with the inflammation process. There is evidence in the scientific literature that prostaglandins act as modulators of the bone resorption process. There is also evidence that certain NSAIDs may to some degree reduce bone resorption [See, for example, the reports on the use of diclofenac sodium by postmenopausal women (Am. J. Medicine, Vol. 96, pp. 349-353, 1994) and naproxen (J. Bone Mineral Res., Vol. 5, pp. 1029-1035, 1990)] in ovariectomized rats. COX-2 inhibitors are disclosed in U.S. Pat. No. 5,663,195 (assigned to Merck and the contents of which are incorporated by reference herein in their entirety) to be useful for the treatment of osteoporosis, prevention of bone loss, periodontal disease, Paget's disease and other ailments. Additionally, U.S. Pat. No. 5,663,195 discloses the usefulness of selective COX-2 inhibitors in inhibiting bone resorption, halting or retarding loss of bone mass, reducing fractures, improving bone repair and preventing or treating osteoporosis and alveolar bone loss due to periodontal disease. Compositions and methods for treatment of periodontal disease comprising selective COX-2 inhibitors are also disclosed in U.S. Pat. No. 5,663,195. The use of bisphosphonates, such as alendronate, for the treatment of periodontal disease in mammals is described in U.S. Pat. No. 5,270,365.

Integrins are also implicated in osteoclast mediated bone resorption. Integrin receptors are heterodimeric transmembrane proteins through which cells attach and communicate with extracellular matrices and other cells [See S. B. Rodan and G. A. Rodan, “Integrin Function In Osteoclasts”, Journal of Endocrinology, Vol. 154, S47-S56 (1997), which is incorporated by reference herein in its entirety]. Bone resorption is mediated by the action of cells known as osteoclasts. Osteoclasts are actively motile cells that migrate along the surface of bone, and can bind to bone, secrete necessary acids and proteases, thereby causing the actual resorption of mineralized tissue from the bone. More specifically, osteoclasts are believed to exist in at least two physiological states, namely, the secretory state and the migratory or motile state. In the secretory state, osteoclasts are flat, attach to the bone matrix via a tight attachment zone (sealing zone), become highly polarized, form a ruffled border, and secrete lysosomal enzymes and protons to resorb bone. The adhesion of osteoclasts to bone surfaces is an important initial step in bone resorption. In the migratory or motile state, the osteoclasts migrate across bone matrix and do not take part in resorption until they again attach to bone.

Integrins are involved in osteoclast attachment, activation and migration. The most abundant integrin in osteoclasts, e.g., in rat, chicken, mouse and human osteoclasts, is an integrin receptor known as αvβ3, which is thought to interact in bone with matrix proteins that contain the RGD sequence. Antibodies to αvβ3 block bone resorption in vitro indicating that this integrin plays a key role in the resorptive process. There is increasing evidence to suggest that αvβ3 ligands can be used effectively to inhibit osteoclast mediated bone resorption in vivo in mammals.

Periodontal disease is a chronic disease that affects large numbers of people [S. Cripps, “Periodontal Disease: Recognition, Interception and Prevention,” Quintessence Publishing Co. (1984)]. Despite the control of periodontal disease achieved by current treatment methods, the risk of bone and resulting tooth loss is still substantial in treated patients. A recent study showed that although, continuous and intermittent non-surgical therapy over a three year period reduced tooth loss by 58% and 48%, respectively, half of the treated patients still suffered tooth loss resulting from bone loss [See P. Hujoel, “Non-Surgical Periodontal Therapy and Tooth Loss. A Cohort Study,” J. Periodontol., Vol. 71, pp. 736-742 (2000)].

There is, therefore, a significant need for an effective treatment of alveolar bone loss associated with advanced periodontitis. The instant invention addresses this problem by providing a combination therapy comprised of a selective COX-2 inhibitor and an αvβ3 receptor antagonist that reduces inflammation and reduces the alveolar bone loss associated with periodontal disease. There is an additional need to have adequate adjunct therapy to prevent or treat alveolar bone loss associated with periodontal surgery. The instant claimed invention may advantageously be used as adjunct therapy in conjunction with or after periodontal surgery to treat alveolar bone loss. When administered as part of a combination therapy, the selective COX-2 inhibitor together with the αvβ3 integrin receptor antagonist provide enhanced therapy treatment options as compared to the administration of either the selective COX-2 inhibitor or the αvβ3 integrin receptor antagonist alone.

It is another object of the present invention to provide pharmaceutical compositions comprising the combination of a selective COX-2 inhibitor and an αvβ3 integrin receptor antagonist that are useful in the methods of the present invention.

It is still a further object of the present invention to provide a method of manufacture of a medicament useful in the treatment of the inflammation and alveolar bone loss associated with periodontal disease. It is yet another object of the present invention to provide a combination therapy that produces superior results in the treatment of periodontal disease compared to the results obtained from administering a selective COX-2 inhibitor or an αvβ3 integrin receptor antagonist alone.

### SUMMARY OF THE INVENTION

The present invention provides for methods of treating the inflammation and alveolar bone loss associated with periodontal disease in a subject in need of such treatment comprising administration of a therapeutically effective amount of an αvβ3 integrin receptor antagonist in combination with a therapeutically effective amount of a selective COX-2 inhibitor. Further the invention provides for pharmaceutical compositions useful in the methods of the present invention, as well as a method of manufacture of a medicament useful to treat periodontal disease.

### DETAILED DESCRIPTION OF THE INVENTION

The present invention is concerned with the combination of an αvβ3 integrin receptor antagonist with a COX-2 inhibitor for the treatment of the inflammation and alveolar bone loss associated with periodontal disease in mammals. This particular combination produces superior results in the treatment of periodontal disease compared to the results from administering a COX-2 inhibitor or an αvβ3 integrin receptor antagonist alone. It is an object of the invention to describe the combination of the two drugs in the treatment of periodontal disease. In addition, it is an object of the instant invention to describe preferred embodiments within each category of compounds which are used as elements in the instant combination. It is a further object of this invention to provide compositions containing each of the compounds for use in the treatment of periodontal disease. It is a still further object of this invention to provide a method of manufacture of a medicament containing the present drug combination which is useful for the treatment of periodontal disease. Further objects will become apparent from a reading of the following description.

The present invention comprises the combination of an αvβ3 integrin receptor antagonist and a selective COX-2 inhibitor for treatment of alveolar bone loss due to periodontal disease. The terms “inhibitor of cyclooxygenase-2,” “cyclooxygenase-2 inhibitor” and “COX-2 inhibitor”, as used herein, embrace compounds which selectively inhibit cyclooxygenase-2 over cyclooxygenase-1. Employing the human whole blood COX-1 assay and the human whole blood COX-2 assay described in C. Brideau et al; Inflamm. Res. 45: 68-74 (1996), herein incorporated by reference, preferably, the compounds have a cyclooxygenase-2 IC₅₀ of less than about 2 μM in human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 10, and preferably of at least 40. The resulting selectivity may indicate an ability to reduce the incidence of common NSAID-induced side effects, such as life threatening ulcers, gastrointestinal toxicity, and impaired renal function.

In the instant combination for the treatment of alveolar bone loss due to periodontal disease, the first element of the combination is a selective COX-2 inhibitor. Selective COX-2 inhibitors have been described in the scientific and patent literature, and reference is made to the following disclosures, the contents of each of which are incorporated by reference herein in their entirety:

(1) J. Talley, Exp. Opin Ther. Patents, “Selective Inhibitors of cyclooxygenase-2,” Vol. 7, pp. 55-62 (1997).

(2) A. Kalgutkar, Exp. Opin Ther. Patents, “Selective Cyclooxygenase-2 inhibitors as non-ulcerogenic anti-inflammatory agents,” Vol. 9, pp. 831-849 (1999).

(3) P. Prasit and D. Riendeau, “Selective Cyclooxygenase-2 Inhibitors,” Annual Reports in Medicinal Chemistry, Volume 32, pp. 211-220 (1997).

Various structural classes of COX-2 inhibitors are disclosed in WO 99/10331, WO 98/21195, WO 98/05639; U.S. Pat. No. 6,025,353; and U.S. Pat. No. 5,741,798; the contents of each of which are incorporated by reference herein in their entirety.

Representative COX-2 inhibitors are also disclosed in the following issued U.S. patents. The Merck (methylsulfonyl)phenyl-2-(5H)-furanone COX-2 inhibitors of the structural formulae I and II, are disclosed in U.S. Pat. No. 5,474,995 and U.S. Pat. No. 6,020,343, respectively, with the definition of substituents, preferred embodiments, and species described therein. These patents provide methods for the preparation of the COX-2 inhibitors of structural formulae I and II.

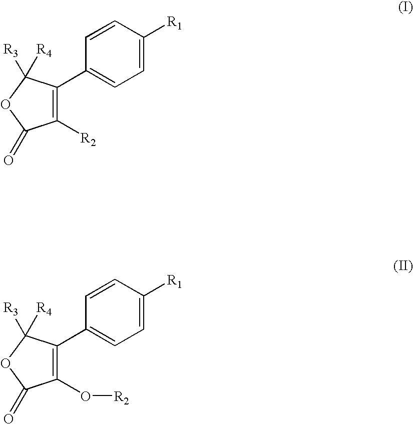

A representative example of a COX-2 inhibitor of structural formula (I) is 3-phenyl-4-(4-(methylsulfonyl)phenyl)-2-(5H)-furanone (rofecoxib) shown below:

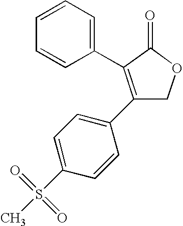

The Merck substituted pyridine COX-2 inhibitors of structural formula (III) are disclosed in U.S. Pat. No. 5,861,419, which is incorporated by reference herein in its entirety. This patent also provides methods for the preparation of such COX-2 inhibitors, with the definition of substituents, preferred embodiments, and species described therein.

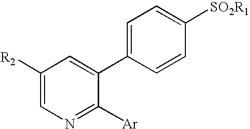

A representative example of a COX-2 inhibitor of structural formula (III) is 5-chloro-3-(4-(methylsulfonyl)phenyl)-2-(2-methyl-5-pyridinyl)pyridine (etoricoxib) shown below:

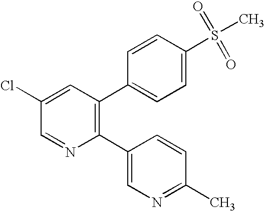

The Searle substituted pyrazolyl benzenesulfonamide COX-2 inhibitors of structural formula (IV) are disclosed in U.S. Pat. No. 5,466,823, which is incorporated by reference herein in its entirety. This patent also provides methods for the preparation of these COX-2 inhibitors, with the definition of substituents, preferred embodiments, and species described therein.

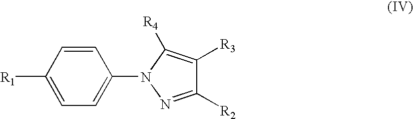

A representative example of a COX-2 inhibitor of structural formula (IV) is 4-[5-(4-methylphenyl)-3-(trifluoromethyl)-1H-pyrazol-1-yl]benzenesulfonamide (celecoxib) shown below:

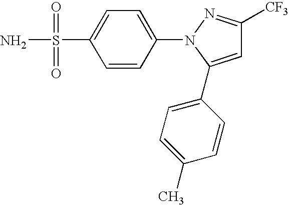

The Searle substituted isoxazole COX-2 inhibitors of structural formula (V) are disclosed in U.S. Pat. No. 5,633,272, which is incorporated by reference herein in its entirety. This patent also provides methods for the preparation of these COX-2 inhibitors, with the definition of substituent, preferred embodiments, and species described therein.

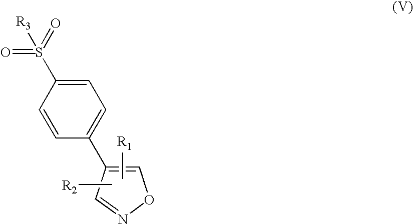

A representative example of a COX-2 inhibitor of structural formula (V) is 4-(5-methyl-3-phenyl4-isoxazolyl)-benzenesulfonamide (valdecoxib) shown below:

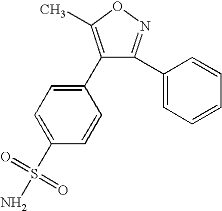

The Searle prodrugs of benzenesulfonamide-containing COX-2 inhibitors of structural formula (VI) are disclosed in U.S. Pat. No. 5,932,598, which is incorporated by reference herein in its entirety. This patent also provides methods for the preparation of these COX-2 inhibitors, with the definition of substituents, preferred embodiments, and species described therein.

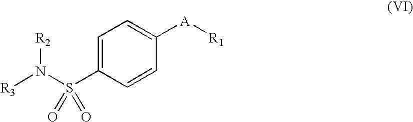

A representative example of a COX-2 inhibitor of structural formula (VI) is N-[[4-(5-methyl-3-phenyl-4-isoxazolyl)phenyl]sulfonyl]propanamide (parecoxib) shown below:

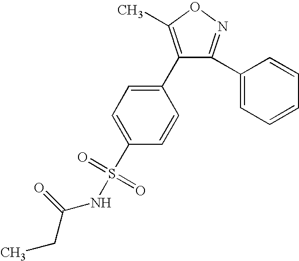

In one embodiment of the combination of the present invention, the selective COX-2 inhibitor is selected from the group consisting of:

3-phenyl-4-(4-(methylsulfonyl)phenyl)-2-(5H)-furanone;

3-(3-fluorophenyl)-4-(4-(methylsulfonyl)phenyl)-2-(5H)-furanone;

3-(3,4-difluorophenyl)-4-(4-(methylsulfonyl)phenyl)-2-(5H)-furanone;

3-(3,4-trichlorophenyl)-4-(4-(methylsulfonyl)phenyl)-2-(5H)-furanone;

3-(3,4-dichlorophenyl)-4-(4-(aminosulfonyl)phenyl)-2-(5H)-furanone;

3-(3-chloro-4-methoxyphenyl)-4-(4-(aminosulfonyl)phenyl)-2-(5H)-furanone;

4-[5-(4-methylphenyl)-3-(trifluoromethyl)-1H-pyrazol-1-yl]benzenesulfonamide;

(5S)-5-ethyl-5-methyl-4-(4-(methanesulfonyl)phenyl)-3-(2-propoxy)-(5H)-furan-2-one;

5,5-dimethyl-4-(4-(methylsulfonyl)phenyl)-3-(2-propoxy)-5H-furan-2-one;

5,5-dimethyl-4-(4-(methylsulfonyl)phenyl)-3-(5-bromopyridin-2-yloxy)-5H-furan-2-one;

5-methyl-4-(4-(methylsulfonyl)phenyl)-3-(2-(propoxy)-5-(2-trifluoroethyl)-5H-furan-2-one;

3-(3-trifluoromethyl)phenoxy-4-(4-(methylsulfonyl)phenyl)-5,5-dimethyl-5H-furan-2-one;

(5R)-3-(3-chloro-4-methoxyphenoxy)-5-ethyl-5-methyl-4-(4-(methylsulfonyl)phenyl)-5H-furan-2-one;

5-chloro-3-(4-(methylsulfonyl)phenyl)-2-(2-methyl-5-pyridinyl)pyridine;

5-chloro-3-(4-(methylsulfonyl)phenyl)-2-(2-ethyl-5-pyridinyl)pyridine;

5-chloro-3-(4-(methylsulfonyl)phenyl)-2-(3-pyridinyl)pyridine;

4-(5-methyl-3-phenyl-4-isoxazolyl)-benzenesulfonamide; and

N-[[4-(5-methyl-3-phenyl-4-isoxazolyl)phenyl]sulfonyl] propanamide;

or a pharmaceutically acceptable salt thereof.

In a class of this embodiment of the present invention, the selective COX-2 inhibitor is selected from the group consisting of:

3-phenyl-4-(4-(methylsulfonyl)phenyl)-2-(5H)-furanone;

4-[5-(4-methylphenyl)-3-(trifluoromethyl)-1H-pyrazol-1-yl]benzenesulfonamide;

4-(5-methyl-3-phenyl-4-isoxazolyl)-benzenesulfonamide;

N-[[4-(5-methyl-3-phenyl-4-isoxazolyl)phenyl]sulfonyl]propanamide;

5-chloro-3-(4-(methylsulfonyl)phenyl)-2-(2-methyl-5-pyridinyl)pyridine; and

(5S)-5-ethyl-5-methyl-4-(4-(methanesulfonyl)phenyl)-3-(2-propoxy)-(5H)-furan-2-one;

or a pharmaceutically acceptable salt thereof.

In the instant combination for the treatment of periodontal disease, the second element of the combination is an αvβ3 integrin receptor antagonist. Selective αvβ3 integrin receptor antagonists have been described, and reference is made to the following disclosures, which are incorporated by reference herein in their entirety: G. Hartman and M. Duggan, “αvβ3 Integrin Antagonists as Inhibitors of Bone Resorption,” Exp. Opin. Invest. Drugs Vol. 9, pp.1281-1291 (2000); and G. Hartman and M. Duggan, “Ligands to the integrin receptor αvβ3,” Exp. Opin. Ther. Patents Vol. 10, pp.1367-1383 (2000).

Compounds, compositions and methods for treatment of treatment of bone resorption comprising αvβ3 integrin receptor antagonists are disclosed in U.S. Pat. No. 6,017,926, which is incorporated by reference herein in its entirety.

In one embodiment of the second element of the combination of the present invention, the αvβ3 integrin receptor antagonist is a compound of the structural formula VII:

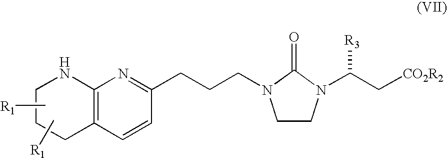

wherein each R¹ is independently selected from the group consisting of hydrogen, C₁₋₄ alkyl, and cyclopropyl; or two R¹ substituents, when on the same carbon atom, are taken together with the carbon atom to which they are attached to form a spirocyclopropyl group;

R² is hydrogen or C₁₋₄ alkyl; and

R³ is a mono- or di-substituted

quinolyl,

pyridinyl, or

pyrimidinyl;

wherein the substituents are each independently hydrogen, halogen, phenyl, C₁₋₄ alkyl, C₃₋₆ cycloalkyl, C₁₋₃ alkoxy, amino, C₁₋₃ alkylamino, di(C₁₋₃) alkylamino, hydroxy, cyano, trifluoromethyl, 1,1,1-trifluoroethyl, trifluoromethoxy, or trifluoroethoxy.

The preparation of the αvβ3 integrin receptor antagonists of structural Formula VII is also disclosed in U.S. Pat. No. 6,017,926.

In a class of this embodiment of the present invention, the αvβ3 integrin receptor antagonist is selected from the group consisting of:

3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;

3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;

3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;

3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and

3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;

or a pharmaceutically acceptable salt thereof.

In a subclass of this embodiment of the present invention, the αvβ3 integrin receptor antagonist is selected from the group consisting of:

3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;

3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid; and

3(S)-(quinolin-3-yl)-3-{2-oxo-3-[3-(5,6,7,8-tetrahydro-[1,8]naphthyridin-2-yl)-propyl]-imidazolidin-1-yl}-propionic acid;

or a pharmaceutically acceptable salt thereof.

In a subclass of this subclass of this embodiment of the combination of the present invention, the αvβ3 integrin receptor antagonist is 3(S)-(6-methoxypyridin-3-yl)-3-{2-oxo-3-[3-(5,6,7,8-tetrahydro-[1,8]naphthyridin-2-yl)-propyl]-imidazolidin-1-yl}-propionic acid and the COX-2 inhibitor is 3-phenyl4-(4-(methylsulfonyl)phenyl)-2-(5H)-furanone.

In yet another subclass of this subclass of this embodiment of the combination of the present invention, the αvβ3 integrin receptor antagonist is 3(S)-(6-methoxypyridin-3-yl)-3-{2-oxo-3-[3-(5,6,7,8-tetrahydro-[1,8]naphthyridin-2-yl)-propyl]-imidazolidin-1-yl}-propionic acid and the COX-2 inhibitor is 4-[5-(4-methylphenyl)-3-(trifluoromethyl)-1H-pyrazol-1-yl] benzenesulfonamide.

In another embodiment of the second element of the combination of the present invention the αvβ3 integrin receptor antagonist is a compound of the structural formula VIII:

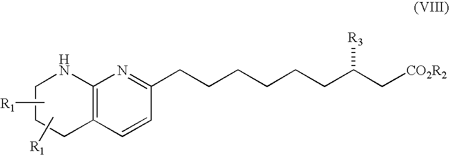

wherein each R¹ is independently selected from the group consisting of hydrogen, C₁₋₄ alkyl, and cyclopropyl; or two R¹ substituents, when on the same carbon atom, are taken together with the carbon atom to which they are attached to form a spirocyclopropyl group;

R² is hydrogen or C₁₋₄ alkyl; and

R³ is aryl wherein aryl is selected from the group consisting of a mono- or disubstituted

quinolyl,

pyridinyl, or

pyrimidinyl;

wherein the substituents are each independently hydrogen, halogen, phenyl, C₁₋₄ alkyl, C₃₋₆ cycloalkyl, C₁₋₃ alkoxy, amino, C₁₋₃ alkylamino, di(C₁₋₃) alkylamino, hydroxy, cyano, trifluoromethyl, 1,1,1-trifluoroethyl, trifluoromethoxy, or trifluoroethoxy.

The preparation of the αvβ3 integrin receptor antagonists of structural Formula VIII is also disclosed in U.S. Pat. No. 6,048,861.

In a class of this embodiment of the combination of the present invention, the αvβ3 integrin receptor antagonist is selected from the group consisting of:

3(S)-(2-methoxy-pyrimidin-5-yl)-9-(5,6,7,8-tetrahydro-[1,8]-naphthyridin-2-yl)-nonanoic acid;

3(S)-(pyrimidin-5-yl)-9-(5,6,7,8-tetrahydro-[1,8]-naphthyridin-2-yl)-nonanoic acid;

3(S)-(2-methyl-pyrimidin-5-yl)-9-(5,6,7,8-tetrahydro-[1,8]-naphthyridin-2-yl)-nonanoic acid; and

3(S)-(quinolin-3-yl)-9-(5,6,7,8-tetrahydro-[1,8]-naphthyridin-2-yl)-nonanoic acid; or a pharmaceutically acceptable salt thereof.

In a subclass of this subclass of this embodiment of the combination of the present invention, the αvβ3 integrin receptor antagonist is 3(S)-(pyrimidin-5-yl)-9-(5,6,7,8-tetrahydro-[1,8]-naphthyridin-2-yl)-nonanoic acid or 3(S)-(2-methyl-pyrimidin-5-yl)-9-(5,6,7,8-tetrahydro-[1,8]naphthyridin-2-yl)-nonanoic acid and the COX-2 inhibitor is 3-phenyl-4-(4-(methylsulfonyl)phenyl)-2-(5H)-furanone.

In yet another subclass of this subclass of this embodiment of the combination of the present invention, the αvβ3 integrin receptor antagonist is 3(S)-(pyrimidin-5-yl)-9-(5,6,7,8-tetrahydro-[1,8]-naphthyridin-2-yl)-nonanoic acid or 3(S)-(2-methyl-pyrimidin-5-yl)-9-(5,6,7,8-tetrahydro-[1,8]naphthyridin-2-yl)-nonanoic acid and the COX-2 inhibitor is 4-[5-(4-methylphenyl)-3-(trifluoromethyl)-1H-pyrazol-1-yl]benzenesulfonamide.

The instant pharmaceutical combination comprising an αvβ3 integrin receptor antagonist in combination with a selective COX-2 inhibitor includes administration of a single pharmaceutical dosage formulation which contains both the αvβ3 receptor antagonist and a COX-2 inhibitor, as well as administration of each active agent in its own separate pharmaceutical dosage formulation. Where separate dosage formulations are used, the αvβ3 receptor antagonist and the COX-2 inhibitor can be administered at essentially the same time, i.e., concurrently, or at separately staggered times, i.e. sequentially. The instant pharmaceutical combination is understood to include all these regimens. Administration in these various ways is suitable for the present combination as long as the beneficial pharmaceutical effect of the αvβ3 receptor antagonist and the COX-2 inhibitor is realized by the patient substantially at the same time. Such beneficial effect is preferably achieved when the target blood level concentrations of each active drug are maintained substantially at the same time. It is preferred that the αvβ3 receptor antagonist and the COX-2 inhibitor be co-administered concurrently on a once-a-day dosing schedule. However, varying dosing schedules, such as the αvβ3 receptor antagonist once or twice a day and the COX-2 inhibitor once, twice or more times per day, is also encompassed herein. A single oral dosage formulation comprised of both an αvβ3 receptor antagonist and the COX-2 inhibitor is preferred. A single dosage formulation will provide convenience for the patient.

In the combination of the present invention, the COX-2 inhibitor may be administered separately at different times during the course of therapy or in conjunction with the αvβ3 integrin receptor antagonist in divided or single combination forms. In addition, the administration of one element of the combination of the present invention may be prior to, concurrent to, or subsequent to the administration of the other element of the combination. The instant invention is therefore to be understood as embracing all such regimes of simultaneous or alternating treatment, and the term “administering” is to be interpreted accordingly. It will be understood that the scope of combinations of the compounds of this invention with other agents useful for treating integrin and cyclooxygenase mediated conditions includes in principle any combination with any pharmaceutical composition useful for treating alveolar bone loss in periodontal disease.

The term integrin receptor antagonist is intended to include all pharmaceutically acceptable salt forms of compounds which have integrin receptor antagonist activity, and therefore the use of such salts is included within the scope of this invention. For use in medicine, the salts of the compounds of this invention refer to non-toxic “pharmaceutically acceptable salts.” Other salts may, however, be useful in the preparation of the compounds according to the invention or of their pharmaceutically acceptable salts. Salts of basic compounds encompassed within the term “pharmaceutically acceptable salts” refer to non-toxic salts of the compounds of this invention which are generally prepared by reacting the free base with a suitable organic or inorganic acid. Representative salts of basic compounds of the present invention include, but are not limited to, the following:

Acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers. Also included within the scope of the invention are polymorphs and hydrates of the compounds of the instant invention.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of periodontal disease with the compound specifically disclosed as an element of the combination or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985.

Some of the compounds described herein may exist as tautomers such as keto-enol tautomers. The individual tautomers as well as mixtures thereof are also encompassed within the scope of compounds of the present invention.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

The elements of the combination of the present invention may be administered by oral, parenteral (e.g., intramuscular, intraperitoneal, intravenous or subcutaneous injection, or implant), buccal, nasal, vaginal, rectal, sublingual, or topical (e.g., ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in the combination in an amount sufficient to produce the desired pharmacologic effect upon the process or condition of periodontal disease.

The pharmaceutical compositions containing the active ingredient suitable for oral administration may be in the form of discrete units such as hard or soft capsules, tablets, troches or lozenges, each containing a predetermined amount of the active ingredient; in the form of a dispersible powder or granules; in the form of a solution or a suspension in an aqueous liquid or non-aqueous liquid; in the form of syrups or elixirs; or in the form of an oil-in-water emulsion or a water-in-oil emulsion. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide a pharmaceutically elegant and palatable preparation.

Solid dosage forms for oral administration include capsules, tablets, pills, powders and granules. In such solid dosage forms, the active compounds are admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets and pills, the dosage forms may also comprise buffering agents.

Tablets containing the active ingredient in admixture with non-toxic pharmaceutically acceptable excipients may also be manufactured by known methods. The excipients used may be for example, (1) inert diluents such as calcium carbonate, lactose, calcium phosphate or sodium phosphate; (2) granulating and disintegrating agents, such as corn starch or alginic acid; (3) binding agents such as starch, gelatin or acacia; and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874 to form osmotic therapeutic tablets for controlled release.

In some cases, formulations for oral use may be in the form of hard gelatin capsules wherein the active ingredient is mixed with an inert solid diluent, for example calcium carbonate, calcium phosphate or kaolin. They may also be in the form of soft gelatin capsules wherein the active ingredient is mixed with water or an oil medium, for example peanut oil, liquid paraffin, or olive oil.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, and perfuming agents.

Aqueous suspensions normally contain the active materials in admixture with excipients suitable for the manufacture of aqueous suspensions. Such excipients may be

1) suspending agents such as sodium carboxymethyl-cellulose, methylcellulose, hydroxypropylmethyl-cellulose, sodium alginate, polyvinyl-pyrrolidone, gum tragacanth and gum acacia;

(2) dispersing or wetting agents which may be

(a) a naturally-occurring phosphatide such as lecithin,

(b) a condensation product of an alkylene oxide with a fatty acid, for example, polyoxyethylene stearate,

(c) a condensation product of ethylene oxide with a long chain aliphatic alcohol, for example, heptadecaethyleneoxycetanol,

(d) a condensation product of ethylene oxide with a partial ester derived from a fatty acid and a hexitol such as polyoxyethylene sorbitol monooleate, or

(e) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soybean and lecithin, (3) esters or partial esters derived from fatty acids and hexitol anhydrides, for example, sorbitan monooleate, (4) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension or solution. The suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention for parenteral administration include sterile aqueous or non-aqueous solutions, suspension, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use. The combination of this invention may also be administered in the form of suppositories for rectal administration. This composition can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Compositions for buccal, nasal or sublingual administration are also prepared with standard excipients well known in the art.

For topical administration the combination of this invention may be formulated in liquid or semi-liquid preparations such as liniments, lotions, applications; oil-in-water or water-in-oil emulsions such as creams, ointments, jellies or pastes, including tooth-pastes; or solutions or suspensions such as drops, mouthwashes, and the like.

The dosage of the active ingredients in the compositions of this invention may be varied. However, it is necessary that the amount of the active ingredient be such that a suitable dosage form is obtained. The selected dosage depends upon the desired therapeutic effect, on the route of administration and on the duration of the treatment. Dosage ranges in the combination for the COX-2inhibitor and the αvβ3 integrin receptor antagonist are approximately one tenth to one times the clinically effective ranges required to induce the desired therapeutic effect, respectively when the compounds are used singly. Generally, dosage levels of the COX-2inhibitor of between about 0.001 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 250 and 500 milligrams of each of the active ingredients for the symptomatic adjustment of the dosage to the patient to be treated. A medicament typically contains from about 0.01 mg to about 500 mg of each of the active ingredients, preferably, from about 1 mg to about 100 mg of each of the active ingredients. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Dosage levels of the αvβ3 of between about 0.001 to 50 mg/kg of body weight daily, preferably about 0.005 to about 25 mg/kg per day, and more preferably about 0.01 to about 10 mg/kg per day are administered to a patient to obtain effective treatment of periodontal disease.

An especially preferred combination is that wherein the αvβ3 integrin receptor antagonist be administered at a dosage rate of about 0.01 to about 10 mg/kg/day, especially about 0.05 to about 5.0 mg/kg/day, and more particularly about 0.1 to about 5 mg/kg/day, and that the integrin receptor antagonist be administered at a dosage level of about 0.001 to about 20 mg/kg/day, especially about 0.005 to about 10 mg/kg/day, and more particularly about 0.01 to about 5 mg/kg/day.

More particularly illustrating the invention is a pharmaceutical composition comprising any of the compounds described above and a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

The test procedures used to measure the efficacy of the combination of the present invention to treat periodontal disease are described below in the following assays. As appreciated by those of skill in the art, the efficacy of a combination within the scope of the invention may be determined by statistical comparison of results achieved in the presence of that combination to that which is achieved in its absence. Alternatives may also be utilized.

Bone Resorption-Pit Assay

When osteoclasts engage in bone resorption, they can cause the formation of pits in the surface of bone that they are acting upon. Therefore, when testing compounds for their ability to inhibit osteoclasts, it is useful to measure the ability of osteoclasts to excavate these resorption pits when the inhibiting compound is present.

Consecutive 200 micron thick cross sections from a 6 mm cylinder of bovine femur diaphysis are cut with a low speed diamond saw (Isomet, Beuler, Ltd., Lake Bluff, Ill.). Bone slices are pooled, placed in a 10% ethanol solution and refrigerated until further use.

Prior to experimentation, bovine bone slices are sonicated twice, 20 minutes each in H₂O. Cleaned slices are placed in 96 well and sterilized by UV irradiation. Prior to incubation with osteoclasts, the bone slices are hydrated by the addition of 0.2 ml αMEM, pH 6.9 containing 1% penicillin/streptomycin.

Long bones from 7-14 day old rabbits (New Zealand White Hare) are dissected, cleaned of soft tissue and placed in αMEM containing 20 mM HEPES. The bones are minced using scissors until the pieces are <1 mm and transferred to a 50 ml tube in a volume of 25 ml. The tube is rocked gently by hand for 60 cycles, the tissue is sedimented for 1 min., and the supernatant is removed. Another 25 ml of medium is added to the tissue and rocked again. The second supernatant is combined with the first. The number of cells is counted excluding erythrocytes (typically ˜2×10⁷ cells/ml). A cell suspension consisting of 5×10⁶/ml in αMEM containing 2% fetal bovine serum, 10 nM 1,25(OH)₂D₃, and penicillin-streptomycin is prepared. 200 ml aliquots are added to bovine bone slices (200 mm×6 mm) and incubated for 2 hrs. at 37° C. in a humidified 5% CO₂ atmosphere. The medium is removed gently with a micropipettor and fresh medium containing test compounds is added to triplicate wells. The cultures are incubated for 72 hrs., and assayed for c-telopeptide (fragments of the α1 chain of type I collagen) by Crosslaps for culture media (Herlev, Denmark).

Test dosage results are compared with controls and resulting IC₅₀ values are determined for each compound tested.

The appropriateness of extrapolating data from this assay to mammalian (including human) disease states is supported by the teaching found in Sato, M., et al., Journal of Bone and Mineral Research, Vol. 5, No. 1, pp. 31-40, 1990, which is incorporated by reference herein in its entirety. This article teaches that certain bisphosphonates have been used clinically and appear to be effective in the treatment of Paget's disease, hypercalcemia of malignancy, osteolytic lesions produced by bone metastases, and bone loss due to immobilization or sex hormone deficiency. These same bisphosphonates are then tested in the resorption pit assay described above to confirm a correlation between their known utility and positive performance in the assay.

Binding Assay (SPAV3)

The αᵥβ₃ integrin is highly expressed in osteoclasts, the bone resorbing cells. The integrin αᵥβ₃ binds RGD containing proteins including osteopontin, which is abundant in bone. The initial step in osteoclast interaction with bone is believed to be αᵥβ₃ binding to its cognate ligands. Since utilizing radioactive proteins is cumbersome, an RGD-mimetic, non-peptide compound which bind to human αᵥβ₃ integrin with high affinity, has been utilized in binding assays. The unavailability of human osteoclasts in quantities needed for biochemical experiments, made it necessary to utilize a human embryonic kidney (HEK) 293 cell line expressing functional human αᵥβ₃ integrin. Duong et al., J. Bone Miner. Res., 8: S378 (1993), describes a system for expressing the human integrin αᵥβ₃. Human αᵥβ₃ integrin was cloned and expressed in HEK 293 cells at a level of 1×10⁶ receptors per cell. The HEK 293-αᵥβ₃ cell line was used in all human integrin-based binding assays in these studies. αᵥβ₃ was purified from stably transfected HEK 293-αᵥβ₃ cells using a GRGDSPK affinity column. Purified αᵥβ₃ integrin was identified by labeling with ¹²⁵I NaI via iodogen, and immunoprecipitation with anti-αᵥβ₃ or -β₁ and -β₅ antibodies. Typically immunoprecipitation was only observed with β₃ antibodies. A potent, RGD-mimetic, non-peptide compound, ¹²⁵I-L-775219, was utilized as ligand. A binding assay (SPAV3) was established utilizing SPA technology. Lyophilized wheatgerm agglutinin SPA beads (0.1 mg in 50 μL binding buffer, 100 mM HEPES, pH 7.2-7.5; 100 mM NaCl; 1 mM Ca²⁺/Mg²⁺) were incubated with 1 μL purified αᵥβ₃ in 24 μL 50 mM octylglucoside in binding buffer and the following were then added sequentially to 96-well plates: 125 μL of binding buffer, 75 μL receptor/beads mixture, 25 μL of test compounds at various doses, and 25 μL of ¹²⁵I-L-775219. Non-specific binding was measured in the presence of 1 μM unlabeled compound and it was usually <10% of total. Radioactivity counting was done by Packard Topcount.

Ocform Assay

Osteoblast-like cells (1.8 cells), originally derived from mouse calvaria, were plated in CORNING 24 well tissue culture plates in αMEM medium containing ribo- and deoxyribonucleosides, 10% fetal bovine serum and penicillin-streptomycin. Cells were seeded at 40,000/well in the morning. In the afternoon, bone marrow cells were prepared from six week old male Balb/C mice as follows:

Mice were sacrificed, tibiae removed and placed in the above medium. The ends were cut off and the marrow was flushed out of the cavity into a tube with a 1 mL syringe with a 27.5 gauge needle. The marrow was suspended by pipetting up and down. The suspension was passed through >100 mm nylon cell strainer. The resulting suspension was centrifuged at 350×g for seven minutes. The pellet was resuspended, and a sample was diluted in 2% acetic acid to lyse the red cells. The remaining cells were counted in a hemacytometer. The cells were pelleted and resuspended at 1×10⁶ cells/mL. 50 μL was added to each well of 1.8 cells to yield 50,000 cells/well and 1,25-dihydroxy-vitamin D₃ (D₃) was added to each well to a final concentration of 10 nM. The cultures were incubated at 37° C. in a humidified, 5% CO₂ atmosphere. 72 h after the addition of bone marrow, test compounds were added with fresh medium containing D₃ to quadruplicate wells. Compounds were added again after 48 h with fresh medium containing D₃. After an additional 48 h., the medium was removed, wells were washed once with PBS, treated with 0.25 mLs 0.05% trypsin, 0.52 mM EDTA for 10 minutes at 37° C. and washed three times with Hepes buffered saline. Plates were blotted dry on paper towels and 0.5 mL substrate solution (dissolve Naphthol AS-BI Phosphate (Sigma # N-2250) in 0.5 mg/ml in 50 mM acetate, 30 mM tartrate, 0.1% Triton X-100, pH 5.5). After 1 hr incubation at 37° C., the reaction was stopped by adding 50 μl of 1 M NaOH. The plates were swirled on an orbital shaker for 10 minutes to dissolve the precipitate, placed on the fluorescent plate reader, and the fluorescence determined at excitation 360 nm, emission 530 nm.

### Examples of Pharmaceutical Formulations

The following are examples of dosage formulations suitable for use in practicing the instant invention. As a specific embodiment of an oral composition of a combination of the present invention, 200 or 400 mg of 3(S)-(6-methoxypyridin-3-yl)-3-{2-oxo-3-[3-(5,6,7,8-tetrahydro-[1,8]naphthyridin-2-yl)-propyl]-imidazolidin-1-yl}-propionic acid, and 12.5 or 25 mg of rofecoxib are formulated with sufficient finely divided lactose to provide a total amount of 580 to 590 mg to fill a size O hard gelatin capsule. As another specific embodiment of an oral composition of a combination of the present invention, 200 mg of 3(S)-(2-methyl-pyrimidin-5-yl)-9-(5,6,7,8-tetrahydro-[1,8]-naphthyridin-2-yl)-nonanoic acid, and 50 or 100 mg of celecoxib are formulated with sufficient finely divided lactose to provide a total amount of 580 to 590 mg to fill a size O hard gelatin capsule.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the patient being treated for periodontal disease. Likewise, the specific pharmacological responses observed may vary according to and depending upon the particular active compound or combination selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method of treating the alveolar bone loss due to periodontal disease which comprises the topical or systemic administration to a subject in need of such treatment a therapeutically effective amount of a cyclooxygenase-2 (COX-2) inhibitor in combination with a therapeutically effective amount of an αvβ3 integrin receptor antagonist.

2. The method of claim 1 wherein the COX-2 inhibitor is selected from the group consisting of:
3-phenyl-4-(4-(methylsulfonyl)phenyl)-2-(5H)-furanone;
3-(3-fluorophenyl)-4-(4-(methylsulfonyl)phenyl)-2-(5H)-furanone;
3-(3,4-difluorophenyl)-4-(4-(methylsulfonyl)phenyl)-2-(5H)-furanone;
3-(3,4-trichlorophenyl)-4-(4-(methylsulfonyl)phenyl)-2-(5H)-furanone;
3-(3,4-dichlorophenyl)-4-(4-(aminosulfonyl)phenyl)-2-(5H)-furanone;
3-(3-chloro-4-methoxyphenyl)-4-(4-(aminosulfonyl)phenyl)-2-(5H)-furanone;
4-[5-(4-methylphenyl)-3-(trifluoromethyl)-1H-pyrazol-1-yl]benzenesulfonamide;
(5S)-5-ethyl-5-methyl-4-(4-(methanesulfonyl)phenyl)-3-(2-propoxy)-(5H)-furan-2-one;
5,5-dimethyl-4-(4-(methylsulfonyl)phenyl)-3-(2-propoxy)-5H-furan-2-one;
5,5-dimethyl-4-(4-(methylsulfonyl)phenyl)-3-(5-bromopyridin-2-yloxy)-5H-furan-2-one;
5-methyl-4-(4-(methylsulfonyl)phenyl)-3-(2-(propoxy)-5-(2-trifluoroethyl)-5H-furan-2-one;
3-(3-trifluoromethyl)phenoxy-4-(4-(methylsulfonyl)phenyl)-5,5-dimethyl-5H-furan-2-one;
(5R)-3-(3-chloro-4-methoxyphenoxy)-5-ethyl-5-methyl-4-(4-(methylsulfonyl)phenyl)-5H-furan-2-one;
5-chloro-3-(4-(methylsulfonyl)phenyl)-2-(2-methyl-5-pyridinyl)pyridine;
5-chloro-3-(4-(methylsulfonyl)phenyl)-2-(2-ethyl-5-pyridinyl)pyridine;
5-chloro-3-(4-(methylsulfonyl)phenyl)-2-(3-pyridinyl)pyridine;
4-(5-methyl-3-phenyl-4-isoxazolyl)-benzenesulfonamide; and
N-[[4-(5-methyl-3-phenyl-4-isoxazolyl)phenyl]sulfonyl] propanamide;
or a pharmaceutically acceptable salt thereof.

3. The method of claim 2 wherein the COX-2 inhibitor is selected from the group consisting of:
3-phenyl-4-(4-(methylsulfonyl)phenyl)-2-(5H)-furanone;
4-[5-(4-methylphenyl)-3-(trifluoromethyl)-1H-pyrazol-1-yl]benzenesulfonamide;
4-(5-methyl-3-phenyl-4-isoxazolyl)-benzenesulfonamide;
N-[[4-(5-methyl-3-phenyl-4-isoxazolyl)phenyl]sulfonyl]propanamide;
5-chloro-3-(4-(methylsulfonyl)phenyl)-2-(2-methyl-5-pyridinyl)pyridine; and
(5S)-5-ethyl-5-methyl-4-(4-(methanesulfonyl)phenyl)-3-(2-propoxy)-(5H)-furan-2-one;
or a pharmaceutically acceptable salt thereof.

4. The method of claim 3 wherein the COX-2 inhibitor is 3-phenyl-4-(4-(methylsulfonyl)phenyl)-2-(5H)-furanone or a pharmaceutically acceptable salt thereof.

5. The method of claim 3 wherein the COX-2 inhibitor is 4-[5-(4-methylphenyl)-3-(tri fluoromethyl)-1H-pyrazol-1-yl]benzenesulfonamide or a pharmaceutically acceptable salt thereof.

6. The method of claim 3 wherein the COX-2 inhibitor is 4-(5-methyl-3-phenyl-4-isoxazolyl)-benzenesulfonamide or a pharmaceutically acceptable salt thereof.

7. The method of claim 3 wherein the COX-2 inhibitor is N-[[4-(5-methyl-3-phenyl-4-isoxazolyl)phenyl]sulfonyl]propanamide or a pharmaceutically acceptable salt thereof.

8. The method of claim 3 wherein the COX-2 inhibitor is 5-chloro-3-(4-(methylsulfonyl)phenyl)-2-(2-methyl-5-pyridinyl)pyridine or a pharmaceutically acceptable salt thereof.

9. The method of claim 3 wherein the COX-2 inhibitor is (5S)-5-ethyl-5-methyl-4-(4-(methanesulfonyl)phenyl)-3-(2-propoxy)-(5H)-furan-2-one or a pharmaceutically acceptable salt thereof.

10. The method of claim 1 wherein the αvβ3 integrin receptor antagonist is a compound of the structural formula (VII): 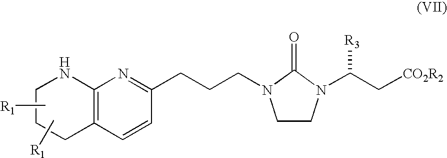
wherein each R¹ is independently selected from the group consisting of hydrogen, C₁₋₄ alkyl, and cyclopropyl; or two R¹ substituents, when on the same carbon atom, are taken together with the carbon atom to which they are attached to form a spirocyclopropyl group;
R² is hydrogen or C₁₋₄ alkyl; and
R³ is aryl wherein aryl is a mono- or disubstituted
quinolyl,
pyridinyl, or
pyrimidinyl;
wherein the substituents are each independently hydrogen, halogen, phenyl, C₁₋₄ alkyl, C₃₋₆ cycloalkyl, C₁₋₃ alkoxy, amino, C₁₋₃ alkylamino, di(C₁₋₃ alkyl)amino, hydroxy, cyano, trifluoromethyl, 1,1,1-trifluoroethyl, trifluoromethoxy, or trifluoroethoxy.

11. The method of claim 10 wherein the αvβ3 integrin receptor antagonist is selected from the group consisting of:
3(S)-(2,3-dihydro-benzofuran-6-yl)-3-{2-oxo-3-[3-(5,6,7,8-tetrahydro-[1,8]-naphthyridin-2-yl)-propyl]-imidazolidin-1-yl}-propionic acid;
3(S)-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
3(S)-(6-ethoxypyridin-3-yl)-3-{2-oxo-3-[3-(5,6,7,8-tetrahydro-[1,8]-naphthyridin-2-yl)-propyl]-imidazolidin-1-yl}-propionic acid;
3(S)-(quinolin-3-yl)-3-{2-oxo-3-[3-(5,6,7,8-tetrahydro-[1,8]naphthyridin-2-yl)-propyl]-imidazolidin-1-yl}-propionic acid; and
3(S)-(4-ethoxy-3-fluorophenyl)-3-{2-oxo-3-[3-(5,6,7,8-tetrahydro-[1,8]-naphthyridin-2-yl)-propyl]-imidazolidin-1-yl}-propionic acid;
or a pharmaceutically acceptable salt thereof.

12. The method of claim 11 wherein the αvβ3 integrin receptor antagonist is 3(S)-(6-methoxypyridin-3-yl)-3-{2-oxo-3-[3-(5,6,7,8-tetrahydro-[1,8]naphthyridin-2-yl)-propyl]-imidazolidin-1-yl}-propionic acid or a pharmaceutically acceptable salt thereof.

13. The method of claim 1 wherein the αvβ3 integrin receptor antagonist is a compound of the structural formula VIII: 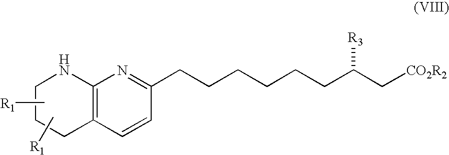
wherein each R¹ is independently selected from the group consisting of hydrogen, C₁₋₄ alkyl, and cyclopropyl; or two R¹ substituents, when on the same carbon atom, are taken together with the carbon atom to which they are attached to form a spirocyclopropyl group;
R² is hydrogen or C₁₋₄ alkyl; and
R³ is a mono- or disubstituted
quinolyl,
pyridinyl, or
pyrimidinyl;
wherein the substituents are each independently hydrogen, halogen, phenyl, C₁₋₄ alkyl, C₃₋₆ cycloalkyl, C₁₋₃ alkoxy, amino, C₁₋₃ alkylamino, di(C₁₋₃ alkyl)amino, hydroxy, cyano, trifluoromethyl, 1,1,1-trifluoroethyl, trifluoromethoxy, or trifluoroethoxy.

14. The method of claim 13 wherein the αvβ3 integrin receptor antagonist is selected from the group consisting of
3(S)-(2-methoxy-pyrimidin-5-yl)-9-(5,6,7,8-tetrahydro-[1,8]-naphthyridin-2-yl)-nonanoic acid;
3(S)-(pyrimidin-5-yl)-9-(5,6,7,8-tetrahydro-[1,8]-naphthyridin-2-yl)-nonanoic acid;
3(S)-(2-methyl-pyrimidin-5-yl)-9-(5,6,7,8-tetrahydro-[1,8]-naphthyridin-2-yl)-nonanoic acid; and
3(S)-(quinolin-3-yl)-9-(5,6,7,8-tetrahydro-[1,8]-naphthyridin-2-yl)-nonanoic acid;
or a pharmaceutically acceptable salt thereof.

15. The method of claim 14 wherein the αvβ3 integrin receptor antagonist is 3(S)-(2-methyl-pyrimidin-5-yl)-9-(5,6,7,8-tetrahydro-[1,8]naphthyridin-2-yl)-nonanoic acid or a pharmaceutically acceptable salt thereof.

16. The method of claim 14 wherein the αvβ3 integrin receptor antagonist is 3(S)-(pyrimidin-5-yl)-9-(5,6,7,8-tetrahydro-[1,8]-naphthyridin-2-yl)-nonanoic acid or a pharmaceutically acceptable salt thereof.

17. The method of claim 12 wherein the COX-2 inhibitor is 3-phenyl-4-(4-(methylsulfonyl)phenyl)-2-(5H)-furanone or a pharmaceutically acceptable salt thereof.

18. The method of claim 12 wherein the COX-2 inhibitor is 4-[5-(4-methylphenyl)-3-(trifluoromethyl)-1H-pyrazol-1-yl]benzenesulfonamide or a pharmaceutically acceptable salt thereof.

19. The method of claim 15 wherein the COX-2 inhibitor is 3-phenyl-4-(4-(methylsulfonyl)phenyl)-2-(5H)-furanone or a pharmaceutically acceptable salt thereof.

20. The method of claim 15 wherein the COX-2 inhibitor is 4-[5-(4-methylphenyl)-3-(trifluoromethyl)-1H-pyrazol-1-yl]benzenesulfonamide or a pharmaceutically acceptable salt thereof.

21. A pharmaceutical composition for the treatment of alveolar bone loss due to periodontal disease which comprises a pharmaceutically acceptable carrier, a therapeutically effective amount of a COX-2 inhibitor and a therapeutically effective amount of an αvβ3 integrin receptor antagonist.

22. The pharmaceutical composition of claim 21 wherein the COX-2 inhibitor is selected from the group consisting of:
3-phenyl-4-(4-(methylsulfonyl)phenyl)-2-(5H)-furanone;
4-[5-(4-methylphenyl)-3-(trifluoromethyl)-1H-pyrazol-1-yl]benzenesulfonamide;
4-(5-methyl-3-phenyl-4-isoxazolyl)-benzenesulfonamide;
N-[[4-(5-methyl-3-phenyl-4-isoxazolyl)phenyl]sulfonyl]propanamide;
5-chloro-3-(4-(methylsulfonyl)phenyl)-2-(2-methyl-5-pyridinyl)pyridine; and
(5S)-5-ethyl-5-methyl-4-(4-(methanesulfonyl)phenyl)-3-(2-propoxy)-(5H)-furan-2-one;
or a pharmaceutically acceptable salt thereof.

23. The pharmaceutical composition of claim 21 wherein the αvβ3 integrin receptor antagonist is selected from the group consisting of:
3(S)-(2-methyl-pyrimidin-5-yl)-9-(5,6,7,8-tetrahydro-[1,8]-naphthyridin-2-yl)-nonanoic acid;
3(S)-(pyrimidin-5-yl)-9-(5,6,7,8-tetrahydro-[1,8]-naphthyridin-2-yl)-nonanoic acid; and
3(S)-(2-methoxypyrimidin-5-yl)-3-{2-oxo-3-[3-(5,6,7,8-tetrahydro-[1,8]-naphthyridin-2-yl)-propyl]-imidazolidin-1-yl}-propionic acid;
or a pharmaceutically acceptable salt thereof.

24. The pharmaceutical composition of claim 23 adapted for topical administration.

25. The use of an αvβ3 integrin receptor antagonist in combination with a COX-2 inhibitor for the preparation of a medicament useful to treat alveolar bone loss due to periodontal disease.

26. The use of claim 25 wherein the αvβ3 integrin receptor antagonist is selected from the group consisting of:
3(S)-(2-methyl-pyrimidin-5-yl)-9-(5,6,7,8-tetrahydro-[1,8]-naphthyridin-2-yl)-nonanoic acid;
3(S)-(pyrimidin-5-yl)-9-(5,6,7,8-tetrahydro-[1,8]-naphthyridin-2-yl)-nonanoic acid; and
3-(6-methoxypyridin-3-yl)-3-{2-oxo-3-[3-(5,6,7,8-tetrahydro-[1,8]-naphthyridin-2-yl)-propyl]-imidazolidin-1-yl}-propionic acid;
or a pharmaceutically acceptable salt thereof.

27. The use of claim 25 wherein the COX-2 inhibitor is selected from the group consisting of:
3-phenyl-4-(4-(methylsulfonyl)phenyl)-2-(5H)-furanone;
4-[5-(4-methylphenyl)-3-(trifluoromethyl)-1H-pyrazol-1-yl] benzenesulfonamide;
4-(5-methyl-3-phenyl-4-isoxazolyl)-benzenesulfonamide;
N-[[4-(5-methyl-3-phenyl-4-isoxazolyl)phenyl]sulfonyl]propanamide;
5-chloro-3-(4-(methylsulfonyl)phenyl)-2-(2-methyl-5-pyridinyl)pyridine; and
(5S)-5-ethyl-5-methyl-4-(4-(methanesulfonyl)phenyl)-3-(2-propoxy)-(5H)-furan-2-one;
or a pharmaceutically acceptable salt thereof.

28. A method of treating alveolar bone loss associated with periodontal disease, in a subject in need thereof, which comprises administering an effective amount of a COX-2 inhibitor in combination with an αvβ3 integrin receptor antagonist according to claim 1 as an adjunct therapy to periodontal surgery.
